(19) United States
(12) Patent Application Publication
(10) Pub. No.: US 2004/0146565 A1
(43) Pub. Date: Jul. 29, 2004

(54) First lipoprotein fraction and therapeutic compositions of same

(75) Inventors: Ronald E. Strohbehn, Nevada, IA (US); Joy M. Campbell, Ames, IA (US); Eric M. Weaver, Story City, IA (US); Jesse Irving Figgins, Ames, IA (US)

Correspondence Address: SCHWEGMAN, LUNDBERG, WOESSNER & KLUTH, P.A., P.O. BOX 2938, MINNEAPOLIS, MN, 55402, US

(73) Assignee: Lauridsen Group Incorporated, Ames, IA

(21) Appl. No.: 10/352,501

(22) Filed: Jan. 28, 2003

(51) Int. Cl. A61K 9/14; A61K 35/16

(52) U.S. Cl. 424/530; 424/489

(57) ABSTRACT

A pharmaceutical composition used as an anti-inflammatory is prepared. The composition comprises a plasma fraction separated from blood plasma and/or in combination with a pharmaceutical carrier. In a preferred method, the plasma fraction is separated from the blood plasma by mixing with from 3% to 10% by weight of the plasma fraction of silica and thereafter centrifuging to allow the plasma fraction to be precipitated out in conjunction with the silica. They are thereafter separated.

### FIELD OF THE INVENTION

The field of this invention relates to a pharmaceutical composition and a method of use of the composition derived from a first lipoprotein fraction of plasma preferably bovine or porcine.

### BACKGROUND OF THE INVENTION

There is a continuing need for the development of medicinals from natural materials. For example, it has been found in the past that certain fractions separated from blood plasma have some therapeutic effects. As an example, a detailed review of the use of intravenous immunoglobulin as drug therapy for manipulating the immune system is described in Vol. 326, No. 2, pp. 107-116, New England Journal of Medicine. The assignee of the present application is involved in continuing research on uses for porcine and bovine blood and blood fractions. Uses for such fractions in the past have been found to be nutrients, and as immune system modulators, see for example co-pending applications of the present inventors entitled Methods and Compositions for Modulating the Immune System of Animals, Ser. No. 09/973,283 filed Oct. 9, 2001 and Ser. No. 09/973,284 filed Oct. 9, 2001.

This application involves discoveries resulting from the continuing effort of the assignee to develop uses for plasma fractions. One of the first fractions removed in normal separation of plasma is a lipoprotein plasma fraction. In the past it has normally been discarded. Though research and investigation of the first lipoprotein plasma fraction, it has now revealed that it does have a surprising and efficacious use never before known.

It is an object of the present invention to provide methods and pharmaceutical compositions for use as an anti-inflammatory for use with humans and animals.

It is another object of the present invention to provide a method of separating a useful first lipoprotein plasma fraction from whole blood plasma which can be used in various pharmaceutical carrier formats as an anti-inflammatory.

Yet, another object of the invention is to provide an effective treatment composition containing a first lipoprotein plasma fraction which at the option of the pharmaceutical formulator can be either an ointment, a cream, a topical spray, or can be dosed orally as tablets, liquid, or as a granular powder or the like.

The method and the manner of accomplishing these and other objectives of the invention will become apparent from the detailed description of the invention which follows.

### SUMMARY OF THE INVENTION

A pharmaceutical composition used as an anti-inflammatory is prepared. The composition comprises a first lipoprotein fraction of plasma separated from blood plasma preferably bovine or porcine and/or in combination with a pharmaceutical carrier. In a preferred method, the first lipoprotein plasma fraction is separated from the blood plasma by mixing with from 3% to 10% by weight of the plasma fraction of silica and thereafter centrifuging to allow the first lipoprotein plasma fraction to be precipitated out in conjunction with the silica which acts as a flocculent. They are thereafter separated.

### DETAILED DESCRIPTION OF THE PREFERRED EMBODIMENT

The first lipoprotein plasma fraction as used herein is a fraction of plasma derived from blood plasma after separation of red cell component. Typically the source of blood is blood of domesticated livestock obtained from meat processing plants, preferably beef, pork or poultry. Anticoagulant is added to the whole blood and then the blood is centrifuged to separate the plasma. It is the separated blood plasma collected from abattoirs, that is the starting material for the first lipoprotein plasma fraction of the present invention.

The plasma, normally obtained in a chilled fashion, usually at a temperature of about 10° C. is mixed with silica at a level of from about 3% by weight of the plasma to about 10% by weight of the plasma, preferably about 5% by weight of the plasma. After thorough mixing, the silica/plasma mixture is centrifuged and the first lipoprotein plasma fraction precipitated out along with the silica. Silica is, of course, well known and commercially available from a variety of reliable sources.

This first step is a flocculation separation step and while silica is preferred, other flocculating agents can be employed. Silica comes in a variety of forms. Colloidal silica and fumed silica may be used. Other flocculents may include other lipid precipitants such as chitosan and activated charcoal.

The mixture of first lipoprotein plasma fraction and flocculent is thereafter diluted with water such that the water-first lipoprotein-fraction-silica mixture is about 40% to 70% of the silica-fraction mixture, preferably about 60%. After the dilution, the pH is adjusted with, for example, 10% sodium hydroxide to a pH of between 10.5 and 11.5. The solution is then stirred for from one hour to 12 hours and the pH adjusted to acidic condition to a pH between 4.3 and 4.6, preferably 4.5. Suitable acids include a lactic acid solution, an acetic acid solution, citric acid, sulfuric and hydrochloric. These can be used at for example 5% to 25% concentration. Eventually the pH is adjusted to between 2.0 and 3.0 while continually stirring.

Thereafter the solution is allowed to react for from 1 hour to 12 hours and thereafter adjusted using a base, for example 10% sodium hydroxide solution to a pH less than 4.0, preferably between 3.2 and 3.4. The mixture is centrifuged again, and the supernatant collected. The supernatant is thereafter concentrated, for example, using membrane dialysis (10,000 molecular weight membranes and de ionized water) to provide a solids concentration of between 8% and 12%. Thereafter, the pH is adjusted again, for example, using 10% sodium hydroxide and/or 2 normal HCl to a pH less than 4.0 preferably between 3.2 and 3.5 to provide a target range of percent solids of from 12% to 16% in the concentrate. It is this concentrate that can be spray dried to provide the dried plasma fraction. Typical spray drying conditions might be 194° C. to 220° C. inlet temperature and between 90° C. and 95° C. outlet temperature to provide a final moisture content below 5%. Other drying conditions may also be used, for example, ambient air drying, oven drying, vacuum drying and freeze drying. If liquid format is to be used, the concentration will be adjusted based on the specific application. Product characteristics both physical and chemical of the dried plasma fraction have been observed. The following product characteristics are noted.

TABLE 1
(Physical)

| TEST | SPECIFICATION |
|---|---|
| Solubility | Hazy with few or no particulates |
| pH (10% Solution) | 2.8 |
| Particle Size/Screen #30 | 100% through |
| Color (Hunter) | White to cream |
| Flavor/Odor | No off odors |
| Foreign Material | None |

TABLE 2
(Chemical)

| | |
|---|---|
| Protein | 85.0% Minimum |
| Moisture | 6.0% maximum |
| Phospholipids | 1-5 mg/g |
| Cholesterol | 1-5 mg/g |
| Triglycerides | 1-5 mg/g |
| Ash | 5.0% maximum |
| Albumin | 40-50% |
| Globulin | 13-25% |
| Endotoxin | 20 EU/mg maximum |
| Calcium¹ | 0.1-17 mg/g |

A plasma fraction, as above prepared and characterized, can be used in a topical application as a cream dispensed with a non-allergenic over-the-counter hand cream. It can be used in an oral application in a powdered dosage form, for example mixed in a cocoa drink mix. It can also be used with other conventionally used pharmaceutical carriers that provide elegant dosage forms, the likes of which are well-known to formulary pharmacists.

The pharmaceutical preparations of the present invention are manufactured in a manner which is itself well known in the art. For example, the pharmaceutical preparations maybe made by means of conventional mixing, granulating, drageemaking, dissolving, lyophilizing processes. The processes to be used will depend ultimately on the physical properties of the active ingredient used.

Suitable excipients are, in particular, fillers such as sugars for example, lactose or sucrose, mannitol or sorbitol, cellulose preparations and/or calcium phosphates, for example, tricalcium phosphate or calcium hydrogen phosphate, as well as binders such as starch, paste, using, for example, maize starch, wheat starch, rich starch, potato starch, gelatin, gum tragacanth, methyl cellulose, hydroxypropylmethylcellulose, sodium carboxymethylcellulose, and/or polyvinyl pyrrolidone. If desired, disintegrating agents maybe added, such as the above-mentioned starches as well as carboxymethyl starch, cross-linked polyvinyl pyrrolidone, agar, alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Dragee cores maybe provided with suitable coating which, if desired, maybe resistant to gastric juices.

For this purpose concentrated sugar solutions maybe used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments maybe added to the tablet or dragee coatings, for example, for identification and in order to characterize different combination of compound doses.

Other pharmaceutical preparations which can be used orally include push-fit capsules made of gelatin, as well as soft, sealed capsules made of gelatin and plasticizer such as glycerol or sorbitol. The push-fit capsules can contain the active compounds in the form of granules which maybe mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers maybe added.

Generally speaking, when mixed in dry form as a granular powder, the amount of the plasma fraction should be from 4% to 18%, preferably from 7% to 10%. When mixed in a topical cream, it should be from 0.5% to 5.0%, preferably 1% to 3%.

The compositions of the present invention have also been found useful toward enhancing viability of cells or tissues in culture or culture-like conditions. When so used, the level should be at from 0.5% to 2% by weight concentration.

The following examples are offered to provide anecdotal evidence of anti-inflammatory therapy.

### EXAMPLES

Six grams of first lipoprotein plasma fraction, as above prepared, was mixed with 453 g of an over-the-counter non-allergenic hand cream. Thereafter, this was used by a series of patients suffering from inflammatory joint soreness, with some of the patients rubbing it on the stiff and sore joints twice daily, and others using it on an as-needed basis. All of the patients noticed a decrease in soreness, stiffness and reported ease of pain.

Some patients suffering from auto-immune diseases such as arthritis and multiple sclerosis were orally dosed under the care and supervision of a doctor. Oral dosing was of a cocoa drink mix with 60 g of the bovine first lipoprotein plasma fraction from spray drying, as above described, mixed with 840 g of a cocoa drink. The dosage amount was 1T with an 8 oz. drink added to luke-warm water at a temperature not to exceed 40° C. Patients taking the drink again noted improvement both in the lack of swelling and ease of pain and observed an increase in joint mobility. One patient, diagnosed with multiple sclerosis has observed dramatic effects. The patient took 2 heaping tablespoons of the bovine first plasma lipid fraction cocoa mix powder in luke-warm in a cup every morning. Within two weeks, the patient described that she noticed more energy while pain and numbness started to decrease. This patient has been treated now for over a year and pain and numbness has decreased by 50% with a notable increase in energy. Doctors in her Neurology Clinic have noticed the difference, commented on her increased energy and observed her notable increase in strength on the left side of her body. Work is now underway to investigate a further supervised clinical trial.

From the above, it can be seen that the invention accomplishes at least all of its stated objectives.

What is claimed is:

1. A pharmaceutical composition useful as an anti-inflammatory comprising:
a first lipoprotein plasma fraction separated from blood plasma.

2. The composition of claim 1 wherein the blood plasma is selected from the group consisting of bovine, porcine and poultry blood plasma.

3. The composition of claim 1 wherein the first lipoprotein plasma fraction is one which precipitated from animal plasma in the presence of a flocculent.

4. The composition of claim 3 wherein the flocculent is from 3% to 10% by weight of the plasma of silica.

5. The composition of claim 4 wherein the silica is used at a level of about 5% by weight of the plasma.

6. The composition of claim 1 wherein the pharmaceutical carrier is selected from the group consisting of tablets, capsules, ampules for oral use, granulated powders, liquids, topical creams, topical ointments and topical sprays.

7. The composition of claim 6 wherein the pharmaceutical carrier is a granulated powder.

8. The composition of claim 7 wherein the first lipoprotein plasma fraction is from 4% to 18% of said granulated powder.

9. The composition of claim 6 wherein the pharmaceutical carrier is a topical cream.

10. The composition of claim 9 wherein the topical cream is from 0.5% to 5% of first lipoprotein plasma fraction.

11. A process of preparing a first lipoprotein plasma fraction which is suitable as an anti-inflammatory, comprising:
providing blood plasma, separated from whole blood; and
separating the first lipoprotein plasma fraction from the balance of the blood plasma.

12. The process of claim 11 wherein an additional step compromises drying the separated first lipoprotein plasma fraction.

13. The process of claim 10 wherein the drying is by a process selected from spray drying, air drying, oven drying, vacuum drying and freeze drying.

14. The process of claim 13 wherein the drying is spray drying.

15. The process of treating a patient for anti-inflammatory condition comprising:
administering to the patient a small but treatment effective amount of a first lipoprotein plasma fraction separated from blood plasma.

16. The process of claim 15 wherein the amount administered is an orally administered dose of from 4% by weight to 18% by weight.

17. The process of claim 16 wherein the amount administered is an oral dose of from 7% by weight to 10% by weight.

18. The process of claim 15 wherein the dose is topical and is from 0.5% by weight to 5.0% by weight.

19. The process of claim 18 wherein the dose is topical and is from 1% by weight to 3.0% by weight.
